(19) United States
(12) Patent Application Publication
(10) Pub. No.: US 2024/0374666 A1
(43) Pub. Date: Nov. 14, 2024

(54) COMPOSITIONS FOR PREVENTING AND/OR REDUCING VIRAL INFECTION

(71) Applicant: Firmenich SA, Satigny (CH)

(72) Inventors: Qi WANG, Shanghai (CN); Dong-Fang CHEN, Shanghai (CN); Yong Ming YUAN, Shanghai (CN); Yongtao WU, Shanghai (CN); Frederic ZUCCA, Shanghai (CN)

(21) Appl. No.: 18/685,391

(22) PCT Filed: Oct. 5, 2022
(86) PCT No.: PCT/EP22/77624

§ 371 Date: Feb. 21, 2024

(30) Foreign Application Priority Data
Oct. 8, 2021 (WO) PCT/CN2021/122538
Nov. 9, 2021 (EP) 21207083.3

(51) Int. Cl. A61K 36/15; A61K 36/03; A61K 36/185; A61K 36/537; A61K 36/61; A61K 36/67; A61K 36/82; A61K 36/9066; A61K 45/06; A61P 31/14; G01N 33/50

(52) U.S. Cl. CPC A61K 36/15; A61K 36/03; A61K 36/185; A61K 36/537; A61K 36/61; A61K 36/67; A61K 36/82; A61K 36/9066; A61K 45/06; A61P 31/14; G01N 33/5091; G01N 2333/165

(57) ABSTRACT

Described herein is a composition for use in the prevention or treatment of a viral infection, where the composition includes an extract, wherein the extract: (i) is a botanical extract; (ii) reduces viral infection into a host cell, with an IC₅₀ of 10 ppm or less; and (iii) has a zeta potential of 10 mV or less. Also described herein are consumer goods and methods of using the composition.

### INTRODUCTION

In recent years there has been an increase in the frequency of the transmission of novel viral infections into the human population. For example, the first large scale transmission of West Nile Virus occurred in the USA around the year 2000, followed by SARS-COV infections in 2003 in Asia, the H1N1 “swine flu” pandemic flu in 2009, the MERS-COV infections in Asia and the USA in 2012, the Zika virus infection in the Americas in 2016, and then the recent global pandemic caused by the SARS-COV-2 starting in 2019.

Viral infections in a population can be addressed using several fundamental therapeutic approaches: (i) reducing the rate of transmission of the virus by social distancing and preventative measures such as face masks and sanitizing surfaces, (ii) reducing the “viral load” in an infected person thus reducing the rate of infection to new hosts, (iii) large scale vaccination to prevent infection or reduce viral load.

A further approach is to prevent viral infection of a host by preventing or reducing the rate of viral infection of the host cell.

Virus-receptor interactions play a key regulatory role in viral host range, tissue tropism, and viral pathogenesis. Viruses utilize elegant strategies to attach to one or multiple receptors, overcome the plasma membrane barrier, enter, and access the necessary host cell machinery. The viral attachment protein can be viewed as the “key” that unlocks host cells by interacting with the “lock”—the receptor—on the cell surface, and these lock-and-key interactions are critical for viruses to successfully invade host cells.

Many common themes have emerged in virus-receptor utilization within and across virus families demonstrating that viruses often target particular classes of molecules in order to mediate these events. Common viral receptors include sialylated glycans, cell adhesion molecules such as immunoglobulin superfamily members and integrins, and phosphatidylserine receptors. The redundancy in receptor usage suggests that viruses target particular receptors or “common locks” to take advantage of their cellular function and also suggests evolutionary conservation. Due to the importance of initial virus interactions with host cells in viral pathogenesis and the redundancy in viral receptor usage, exploitation of these strategies would be an attractive target for new antiviral therapeutics.

Agents which seek to prevent or reduce viral interaction with the host cell receptor are typically pharmaceutical type compounds such as New Chemical Entities (NCEs) or modulating biological moieties such as monoclonal antibodies. However, such compounds suffer from the disadvantage that they take a long time to develop, they are expensive, and they are not “natural” to the environment meaning they can be consumer reluctance for adoption.

As evidenced by the recent global outbreak of SARS-COV-2 and the associated catastrophic impact on human society, there is an urgent need to identify agents which prevent or treatment viral infection but which are also natural.

From this perspective the present inventors chose to screen known food and flavoring botanical extracts to identify composition which can reduce virus binding to cell receptors.

### SUMMARY OF THE INVENTION

Hence an aspect of the invention provides a composition for use in the prevention or treatment of a viral infection, wherein the composition comprises an extract, wherein said extract:
(i) is a botanical extract;
(ii) reduces viral infection into a host cell, with an IC₅₀ of 10 ppm or less; and,
(iii) has a zeta potential of 10 mV or less.

An embodiment of the invention is wherein the composition comprises an extract from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Laminaria species (preferably Laminaria digitata, Laminaria japonica and/or Laminaria ephemera), Curcuma longa, Camellia sinensis (preferably Camellia sinensis var. assamica), Artemisia species (preferably Artemisia capillaris, Artemisia scoparia, Artemisia dracunculus, Artemisia nakaii and/or Artemisia pubescens), Geum aleppicum and Salvia species (preferably Salvia miltiorrhiza).

An embodiment of the invention is wherein the composition comprises an extract having a zeta potential of 0 mV or less. Preferably in this embodiment the extract is from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Curcuma longa, Artemisia species (preferably Artemisia capillaris, Artemisia scoparia, Artemisia dracunculus, Artemisia nakaii and/or Artemisia pubescens), Geum aleppicum and Salvia species (preferably Salvia miltiorrhiza).

A further embodiment of the invention is wherein the composition comprises an extract having a zeta potential of −10 mV or less. Preferably in this embodiment the extract is from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Curcuma longa,

An embodiment of the invention is wherein the virus infection is caused by a coronavirus. Preferably the coronavirus is SARS-COV-2.

An embodiment of the invention is wherein the extract is at a concentration of 10 ppm or more.

An embodiment of the invention is wherein said composition further comprises one or more of the following ingredients: antiviral agents, antibacterial agents, minerals, mineral salts, and/or vitamins, pharmaceutically acceptable excipients.

An embodiment of the invention is wherein said composition is prepared in a therapeutic form as liquid, cream, lotion, liniment, ointment, gel, paste, tonic, tincture, unguent, topical, oral pill, tablet, capsule, lip balm, spray, or combinations thereof.

A further aspect of the invention provides a consumer product comprising a composition according to the invention.

An embodiment of the invention is wherein the consumer product comprises fabric care product, body-care product, cosmetic preparation, skincare product, air care product or home care product, or wherein the perfumery consumer product is a fine perfume, a splash or eau de perfume, a cologne, a shave or an after-shave lotion, a liquid or solid detergent, a fabric softener, a fabric refresher, an ironing water, a shampoo, a hair shaping product, a dental care product, a disinfectant, an intimate care product, a hair spray, a vanishing cream, a deodorant or antiperspirant, hair remover, tanning or sun product, nail products, skin cleansing, a makeup, a perfumed soap, shower or bath mousse, oil or gel, or a foot/hand care products, a hygiene product, an air freshener, a “ready to use” powdered air freshener, a mold remover, furnisher care, wipe, a dish detergent or hard-surface detergent, a leather care product, a car care product, or a product applied on face masks or other personal protective equipment (essential oil diffuser clips for face masks air freshener, etc.).

An embodiment of the invention is wherein the consumer product comprise beverages including alcoholic drinks (beer, cider, FABs, spirit, wine, etc.), carbonated soft drinks, other soft drinks (bottles water, flavored water, energy drinks, functional drinks, sport drinks, etc.), Ready To Drink (tea, coffee, etc.), hot drinks (coffee, tea, etc.), juices and nectars, Powder Soft Drinks (fruit drink, tea, etc.); and sweet goods including bakery (biscuits, snacks, etc.), cereals, confectioneries (candies, jellies, tablets, gums, chewing gums, chocolate, etc.), dairies (milk, yoghurt and fruit preparations, dairy drinks, etc.), desserts (ice cream, fruits and preserves, etc.), dietary and nutrition (meal replacements, protein bars, protein drinks, etc.); and oral care products including toothpastes and mouthwashes; and savory goods including alternative proteins (cheese analogs, veg proteins products, etc.), prepared meals (processed chesses, ready meals, etc.), snacks, meat proteins (processed meat, sausages, etc.), noodles and soups.

A further aspect of the invention provides a method of treatment or prevention viral infection, the method comprising the application to the oral and/or nasal cavity of a person in need thereof a composition according to any preceding claim.

A further aspect of the invention provides a method of identifying an extract having use in the treatment or prevention viral infection comprising assaying whether an extract reduces pseudovirus entry in a cell using a pseudovirus entry assay.

A further embodiment of the invention is wherein the method further comprises an ELISA assay or SPR assay of the extract.

### DESCRIPTION OF THE INVENTION

The present inventors developed an assay which identifies food, flavoring or perfumery botanical extracts that reduce SARS-COV-2 binding to human receptor ACE2. From a screen of over 500 such botanical extracts, they surprisingly identified 11 botanical extracts which modulate viral infection of the host cells.

Since these food, flavoring and perfumery, and medicinal botanical extracts are well perceived by the public and have no known side effect issues, the invention claims use of the botanical extracts in compositions to prevent or reduce viral infection.

Hence an aspect of the invention provides a composition for use in the prevention or treatment of a viral infection, wherein the composition comprises an extract, wherein said extract:
(i) is a botanical extract;
(ii) reduces viral infection into a host cell, with an IC₅₀ of 10 ppm or less; and,
(iii) has a zeta potential of 10 mV or less.

The extract to be used in the composition is a botanical extract. Botanical extracts are commonly used as food, flavoring, traditional medicines and/or perfumery ingredients and hence are well known and tolerated to the consumer. Consequently, many of them often have little regulatory barriers to market. Hence they are preferred for use the prevention or treatment of viral infections.

An embodiment of the invention is wherein the composition comprises an extract from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Laminaria species (preferably Laminaria digitata, Laminaria japonica and/or Laminaria ephemera), Curcuma longa, Camellia sinensis (preferably Camellia sinensis var. assamica), Artemisia species (preferably Artemisia capillaris, Artemisia scoparia, Artemisia dracunculus, Artemisia nakaii and/or Artemisia pubescens), Geum aleppicum and Salvia species (preferably Salvia miltiorrhiza).

Botanical extracts are well known and readily sourced commercial commodities. By “extract” we include that the unprocessed botanical source material is processed using known techniques to prepare the desired extract, for example using solvents, supercritical CO₂ technologies, microwave, steam, and other such methods.

Examples of all extracts which can be used in present invention can be obtained from Firmenich SA (www.firmench.com).

The extract to be used in the composition reduces viral infection into a host cell, with an IC₅₀ 10 ppm or less, preferably an IC₅₀ 7 ppm or less, more preferably an IC₅₀ of 4 ppm or less.

Provided herein are methods by which the reduction of viral infection into a host cell can be measured. The use of such methods is known in the field of virology and can be readily adopted by the skilled person to determine if an extract reduces viral infection and hence is an extract which can be used in the composition of the invention.

IC₅₀ measurement is a well-known measure of the potency of a substance in inhibiting a specific biological function, in this case viral infection into a host cell. It is a quantitative measure which indicates how much of a particular inhibitory substance, in this case a botanical extract, is needed to inhibit in vitro a given biological process by 50%. The calculation of IC₅₀ measurements for botanical extracts can be readily performed by the skilled person using the methods disclosed herein and known in the art.

The inventors calculated the IC₅₀ value of many hundreds of food, flavoring and perfumery botanical extracts. They identified that a IC₅₀ value of 10 ppm or less is a “cut off” value such that any higher value indicate that the assayed botanical extract would not be capable of preventing or treating a viral infection. Preferably the IC₅₀ value is 7 ppm or less, more preferably an IC₅₀ of 4 ppm or less.

The extract to be used in the composition reduces viral infection into a host cell has a zeta potential of 10 mV or less.

Zeta potential measures the net electrical charge inherent in a substance. This charge is important since it can influence mechanical or functional characteristics of a substance. The charge can be measured via Zeta potential values measured by Zetasizer by dispersing the extract or extract solution into water phase (under a neutral pH value). The zeta potential value may influence stability of colloids formed in a composition comprising the extract.

With respect to their potential to prevent or treat viral infection, while not wishing to be bound by any specific scientific reasoning, the inventors speculate that the utility of an extract as an agent to prevent or treat viral infection may be a function of charge interaction between components in the extract and the site of infection into the host cell. Hence a lower zeta potential may reduce viral infection into a host cell by binding to, or masking, the receptor on the cell surface which is targeted by the viral particle to mediate infection.

An embodiment of the invention is wherein the composition comprises an extract having a zeta potential of less than 0 mV. Preferably in this embodiment the extract is from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Curcuma longa, Artemisia species (preferably Artemisia capillaris, Artemisia scoparia, Artemisia dracunculus, Artemisia nakaii and/or Artemisia pubescens), Geum aleppicum and Salvia species (preferably Salvia miltiorrhiza).

A further embodiment of the invention is wherein the composition comprises an extract having a zeta potential of −10 mV or less. Preferably in this embodiment the extract is from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Curcuma longa,

The first aspect of the invention states that the claimed composition can be used for the prevention or treatment of a viral infection.

By “prevents” we include that the claimed composition prevents, reduces, or interrupts viral infection into a host cell by at least 50% to when the composition is not used. By “treats” we include where the claimed composition prevents, reduces or interrupts viral infection into a secondary host cell in the subject, i.e. replication within the host organism following the initial infective event and location. “Treat” also include where the claimed composition reduces the severity of the consequences of the viral infection, e.g. destruction of more host cells.

Preferably the composition of the invention reduces or interrupts viral infection into a host cell.

### Viral Infection

The present invention provides a composition for use in the prevention or treatment of a viral infection.

By “viral infection” we include infections caused by different classes of viral agents. The extracts used in the composition of the present invention were identified by assays which measure a reduction in viral binding to human receptor ACE2.

Hence an embodiment of the invention is wherein the composition of the invention is used to prevent or treat of a viral infection caused by viral binding to the human ACE2 receptor.

Assays to measure whether a virus binding to the human ACE2 receptor are well known in the art. For example, ELISA, surface plasmon resonance (SPR), Biolayer interferometry (BLI), and pseudovirus entry assays are adopted for measuring viral-cell binding properties, viral entry processes, and for studying and screening viral entry inhibitors (Bao et al. Nature, 2020, 583 (7818): 830-833; Walls, et al. Cell, 2020, 16, 181 (2): 281-292.e6; Shang, et al. PNAS, 2020, 26, 117 (21): 11727-11734; Wang, et al. Phytomedicine, 2020, 79, 153333).

Furthermore, the applicant has provided information in the accompanying Examples section of the application of assays which would allow the skilled person to identify whether a virus binds to the human ACE2 receptor.

ACE2 is considered as a hot spot for viral entry. NL63 coronavirus (NL63-CoV) and SARS coronavirus (SARS-COV) which bind to the human ACE2 receptor are well known in the art (Wu et al. Journal of Virology 2011, 85 (11): 5331-5337).

An embodiment of the invention is wherein the composition is for use in the prevention or treatment of a viral infection caused by a coronavirus group of RNA viruses.

Coronaviruses are a group of viruses well known since at least the 1920s. They large, roughly spherical particles with unique surface projections. Their size is highly variable with average diameters of 80 to 120 nm. Extreme sizes are known from 50 to 200 nm in diameter. The total molecular mass is on average 40,000 kDa. They are enclosed in an envelope embedded with a number of protein molecules.

The spikes are the most distinguishing feature of coronaviruses and are responsible for the corona- or halo-like surface. On average a coronavirus particle has 74 surface spikes. Each spike is about 20 nm long and is composed of a trimer of the S protein. The S proteins have the receptor-binding domains (RBD) and mediate the key step of viral entry into the target host cells via binding to the receptor proteins on the surface of the host cells.

Preferably the coronavirus is SARS-COV-2, SARS-COV, HCOV-NL63, MERS-COV, HCoV-OC43, HCoV-HKU1, HCOV-229E, MERS spike. Preferably the receptor of SARS-CoV-2, SARS-COV, HCOV-NL63, Civet SARS-COV, and BatSARS-CoVr-WIV1 spikes is ACE2. Preferably the receptor of HCoV-229E spike is ANPEP (alanyl aminopeptidase), which is also named as CD13 or hAPN (aminopeptidase N). Preferably the receptor of MERS spike is DPP4 (dipeptidyl peptidase 4), which is also named as CD26. Preferably the receptors of HCoV-HKU1 and HCoV-OC43 spikes are decorated by 9-O-acetylated sialoglycans (Millet, et al. FEMS Microbiology Reviews, 2021, 45 (3), fuaa057).

Preferably the viral infection is caused by SARS-COV-2, SARS-COV, HCoV-NL63, Civet SARS-COV, and BatSARS-CoVr-WIV1 which adopt ACE2 as receptor.

The inventors have also examined the various variants of SARS-COV-2 which have appeared since the earliest identified pathogenic strain of this virus. They have shown in particular that the extracts used in the composition of the invention are effective at preventing the original virus. A preferred embodiment of the invention is wherein the SARS-COV-2 is SARS-COV-2 delta variant.

### Extracts for Use in the Composition of the Invention

An embodiment of the invention is wherein the composition comprises an extract from one or more of the following: Pine species (preferably Pinus sylvestris, Pinus virginiana and/or Pinus thunbergia), Eucalyptus species (preferably Eucalyptus globulus, Eucalyptus maidenii, and/or Eucalyptus smithii), Ribes nigrum, Cistus species (preferably Cistus ladanifer, C. ladanifer subsp. Mauritianus, C. ladanifer subsp. sulcatus Cistus palhinhae, Cistus criticus and/or Cistus laurifolius), Piper nigrum, Laminaria species (preferably Laminaria digitata, Laminaria japonica and/or Laminaria ephemera), Curcuma longa, Camellia sinensis (preferably Camellia sinensis var. assamica), Artemisia species (preferably Artemisia capillaris, Artemisia scoparia, Artemisia dracunculus, Artemisia nakaii and/or Artemisia pubescens), Geum aleppicum and Salvia species (preferably Salvia miltiorrhiza). An embodiment of the invention is wherein the composition comprises an extract from Pine species (preferably Pinus sylvestris, Pinus virginiana and/or Pinus thunbergia).

Preferably the present invention relates to the use of a botanical extract from Pinus sylvestris registered under CAS 8023-99-2 which is commercially available from many sources.

“Pinus sylvestris” is a botanical extract used in flavor and fragrance industry. It is a complex absolute comprising many compounds, including cis-abietal, beta-pinene, alpha-pinene, abietic acid, etc. It is prepared from the leaves (needles) of pine plants (the most relevant species Pinus sylvestris is native to Albania, Altay, Amur, Austria, Baltic States, Belarus, Belgium, Bulgaria, Buryatia, Central European Rus, Chita, Czechoslovakia, East European Russia, Finland, France, Germany, Great Britain, Greece, Hungary, Inner Mongolia, Irkutsk, Italy, Kazakhstan, Khabarovsk, Krasnoyarsk, Krym, Manchuria, Mongolia, New York, North Caucasus, North European Russia, Northwest European R, Norway, Poland, Romania, Spain, Sweden, Switzerland, Transcaucasia, Turkey, Tuva, Ukraine, West Siberia, Yakutskiya, Yugoslavia, and introduced into Denmark, Iceland, Illinois, Ireland, Korea, Netherlands, Portugal, Turkey-in-Europe, Vermont). The absolute is commonly used for flavoring and perfumery purposes.

In a preferred embodiment the amount of Pine species extract (preferably Pinus sylvestris) in the composition of the invention is 44 ppm or more. Preferably the amount of Pine species extract (preferably Pinus sylvestris) in the composition of the invention is less than 500000 ppm.

An embodiment of the invention is wherein the composition comprises an extract from Eucalyptus plants (preferably Eucalyptus globulus). Preferably the present invention relates to the use of a botanical extract from Eucalyptus globulus registered under CAS 8000-48-4 which is commercially available from many sources.

“Eucalyptus globulus” is a botanical extract used in flavor and fragrance industry. It is a complex absolute comprising many compounds, including eucalyptol, aromadendrene, globulol, pinocarvone, etc. It is prepared from the leaves of Eucalyptus globulus (Eucalyptus globulus is native to New South Wales, Tasmania, Victoria, and introduced into Albania, Azores, Baleares, Bangladesh, Bolivia, California, Canary Is., China South-Central, China Southeast, Costa Rica, East Aegean Is., Easter Is., Ecuador, El Salvador, Eritrea, Ethiopia, France, Free State, Great Britain, Guatemala, India, Ireland, Italy, Juan Fernandez Is., Kenya, Leeward Is., Lesser Sunda Is., Madeira, Mexico Southeast, Mexico Southwest, Morocco, New Zealand North, New Zealand South, Pakistan, Panamá, Paraguay, Peru, Portugal, Rwanda, Sardegna, Sicilia, Spain, Spain, St. Helena, Tanzania, Transcaucasus, Uganda, Windward Is., Zimbabwe). The absolute is commonly used for flavoring and perfumery purposes. By “Eucalyptus globulus” we include material registered under CAS 8000-48-4 which is commercially available from many sources.

In a preferred embodiment the amount of Eucalyptus globulus extract in the composition of the invention is 51 ppm or more. Preferably the amount of Eucalyptus globulus extract in the composition of the invention is less than 640000 ppm.

An embodiment of the invention is wherein the composition comprises an extract from Ribes nigrum. By “Ribes nigrum” we include material registered under CAS 97676-19-2 or 68606-81-5 which are commercially available from many sources.

“Ribes nigrum” is a botanical extract used in flavor and fragrance industry. It is a complex absolute comprising many compounds, including quercetin, glycitein, etc. It is prepared from the buds of Ribes nigrum (Ribes nigrum is native to Altay, Baltic States, Belarus, Belgium, Bulgaria, Buryatia, Central European Rus, Chita, Czechoslovakia, Denmark, East European Russia, Finland, France, Germany, Great Britain, Irkutsk, Kazakhstan, Krasnoyarsk, Netherlands, North European Russi, Northwest European R, Norway, Poland, Romania, Sweden, Tadzhikistan, Turkmenistan, Tuva, Ukraine, West Siberia, Yakutskiya, Yugoslavia, and introduced into Austria, Connecticut, Falkland Is., Hungary, Illinois, Inner Mongolia, Ireland, Italy, Korea, Magadan, Maine, Manchuria, Maryland, Massachusetts, Michigan, Minnesota, New Brunswick, New Hampshire, New Jersey, New York, Newfoundland, Nova Scotia, Ohio, Ontario, Primorye, Prince Edward I., Quebec, Switzerland, Turkey, Uzbekistan, Vermont, West Himalaya, Wisconsin, Xinjiang). The absolute is commonly used for flavoring and perfumery purposes.

In a preferred embodiment the amount of Ribes nigrum extract in the composition of the invention is 37 ppm or more.

An embodiment of the invention is wherein the composition comprises an extract from Cistus species (preferably Cistus ladanifer, C. ladanifer subsp. Mauritianus, C. ladanifer subsp. sulcatus Cistus palhinhae, Cistus criticus and/or Cistus laurifolius).

Preferably the present invention relates to the use of a botanical extract from Cistus ladanifer registered under CAS 8016-26-0 which is commercially available from many sources.

“Cistus ladanifer” is a botanical extract used in flavor and fragrance industry. It is a complex absolute comprising many compounds, including alpha-pinene, trans-pinocarveol, verbenone, bornyl acetate, borneol, 4-terpinenol, etc. It is prepared from the leaves and twigs of Cistus ladanifer (Cistus ladanifer is native to France, Morocco, Portugal, Spain, and introduced into California, Canary Is., Cyprus, Madeira, New Zealand South). The absolute is commonly used for perfumery purposes. By “Cistus ladanifer” we include material registered under CAS 8016-26-0 which is commercially available from many sources.

In a preferred embodiment the amount of Cistus species extract (preferably Cistus ladanifer) in the composition of the invention is 26 ppm or more. Preferably the amount of Cistus species extract (preferably Cistus ladanifer) in the composition of the invention is less than 435000 ppm.

An embodiment of the invention is wherein the composition comprises an extract Piper nigrum. Preferably the present invention relates to the use of a botanical extract from Piper nigrum registered under CAS 8006-82-4 or 84929-41-9 which are commercially available from many sources.

“Piper nigrum” is a botanical extract used in flavor and fragrance industry. It is a complex oil comprising many compounds, including limonene, carene, beta-pinene, alpha-pinene, piperine, alpha-copaene, piperanine, etc. It is prepared from the dried pepper berries of Piper nigrum (Piper nigrum is native to India and introduced into Andaman Is., Assam, Bangladesh, Benin, Cambodia, Cameroon, China South-Central, China Southeast, Cook Is., Costa Rica, Cuba, Dominican Republic, East Himalaya, French Guiana, Guinea, Gulf of Guinea Is., Haiti, Honduras, Laos, Leeward Is., Mauritius, Mexico Gulf, Nicobar Is., Philippines, Puerto Rico, Réunion, Seychelles, Sri Lanka, Thailand, Trinidad-Tobago, Venezuela, Vietnam, Windward Is.). The oil is commonly used for flavoring and perfumery purposes. By “Piper nigrum” we include material registered under CAS 8006-82-4 or 84929-41-9 which are commercially available from many sources.

In a preferred embodiment the amount of Piper nigrum extract in the composition of the invention is 10 ppm or more.

An embodiment of the invention is wherein the composition comprises a mixture of extracts from Laminaria digitata and Macrocystis pyrifera.

In a preferred embodiment the mixture of extracts from Laminaria digitata and Macrocystis pyrifera in the composition of the invention is 12 ppm or more.

Preferably the present invention relates to the use of a botanical extract from Laminaria digitata registered under CAS 1207666-16-7 which is commercially available from many sources.

Laminaria digitata is a botanical extract used in flavor and fragrance industry. It is a complex extract comprising many compounds but not limited, including L-pyroglutamic acid, proline, nicotinic acid, mannitol, etc. It is prepared from the seaweed species Laminaria digitata which occurs in the north west Atlantic from Greenland south to Cape Cod and in the north east Atlantic from northern Russia and Iceland south to Franc. By “Laminaria digitata” we include material registered under CAS 1207666-16-7 which is commercially available from many sources.

In a preferred embodiment the amount of Laminaria digitata extract in the composition of the invention is 12 ppm or more.

An embodiment of the invention is wherein the composition comprises an extract from Curcuma longa. Preferably the present invention relates to the use of a botanical extract from Curcuma longa registered under CAS 8024-37-1 or 84775-52-0 which are commercially available from many sources.

“Curcuma longa” is a material used in flavor and fragrance industry. It is a complex oleoresin comprising many compounds, including alpha-curcumene, zingiberene, tumerone, curlone, Ar-turmerone, curcumin, etc. It is prepared from the roots of Curcuma longa (Curcuma longa is native to India and introduced into Andaman Is., Assam, Bangladesh, Belize, Borneo, Cambodia, Caroline Is., China South-Central, China Southeast, Congo, Cook Is., Costa Rica, Cuba, Dominican Republic, East Himalaya, Easter Is., Fiji, Gilbert Is., Guinea-Bissau, Gulf of Guinea Is., Haiti, Hawaii, Ivory Coast, Jawa, Leeward Is., Lesser Sunda Is., Malaya, Mauritius, Myanmar, New Caledonia, New Guinea, Nicobar Is., Philippines, Pitcairn Is., Puerto Rico, Queensland, Réunion, Samoa, Society Is., Sri Lanka, Sumatera, Taiwan, Thailand, Tibet, Tonga, Trinidad-Tobago, Vietnam, Windward Is.). The oleoresin is commonly used for flavoring purposes.

In a preferred embodiment the amount of Curcuma longa extract in the composition of the invention is 18 ppm or more.

An embodiment of the invention is wherein the composition comprises an extract from Camellia sinensis (preferably Camellia sinensis var. assamica, the Puer Tea). Preferably the present invention relates to the use of a botanical extract from Camellia sinensis registered under CAS 68916-73-4 or 84650-60-2 which are commercially available from many sources.

“Camellia sinensis” is a botanical extract used in flavor and fragrance industry. It is a complex extract comprising many compounds, including caffeine, kaempferol, quercetin, myricetin, epicatechin, (−)-epigallocatechin, kaempferol-7-o-glucoside, quercetin-3β-D-glucoside, 7-methylxanthine, theobromine, caffeine, D-serine, L-aspartic acid, leucine, DL-arginine, etc. It is prepared from the leaves of Camellia sinensis (Camellia sinensis is native to China South-Central, China Southeast, and introduced into Andaman Is., Argentina Northeast, Assam, Bangladesh, Borneo, Cambodia, China North-Central, East Himalaya, Ethiopia, Gulf of Guinea Is., Japan, Korea, Laos, Mauritius, Myanmar, Nepal, Réunion, Seychelles, Sri Lanka, Tibet, Trinidad-Tobago, Turkey, Vietnam). The extract is commonly used for flavoring purposes. By “Camellia sinensis” we include material registered under CAS 68916-73-4 or 84650-60-2 which are commercially available from many sources.

In a preferred embodiment the amount of Camellia sinensis extract in the composition of the invention is 67 ppm or more.

An embodiment of the invention is wherein the composition comprises an extract from Artemisia species (preferably Artemisia capillaris, Artemisia scoparia, Artemisia dracunculus, Artemisia nakaii and/or Artemisia pubescens). Preferably the present invention relates to the use of a botanical extract from Artemisia capillaris registered under CAS 223747-93-1 which is commercially available from many sources.

“Artemisia capillaris” is a material used in traditional Chinese medicine. It is a complex extract comprising many compounds, including camphor, α-thujone, β-thujone, sabinene, α-terpinene, eucalyptol, 6,7-dimethylsculetin, α-pinene, capillin, capillene, capillanol, capillarisin, azelaic acid, quercetin, 3-quinic acid, luteolin, etc.

In a preferred embodiment the amount of Artemisia species extract in the composition of the invention is 9 ppm or more.

An embodiment of the invention is wherein the composition comprises an extract from Geum aleppicum. Preferably the present invention relates to the use of a botanical extract from Geum aleppicum which is commercially available from many sources.

“Geum aleppicum” is a material used in traditional Chinese medicine. It is a complex extract comprising many compounds, including potengrtilloside A, palmitic acid, β-sitosterol, benzoic acid, gallate acid, salicylic acid, vanillin, 3,4,5-trihydroxybenzoic dehyde, etc.

In a preferred embodiment the amount of Geum aleppicum extract in the composition of the invention is 22 ppm or more.

An embodiment of the invention is wherein the composition comprises an extract from Salvia plants (preferably Salvia miltiorrhiza). Preferably the present invention relates to the use of a botanical extract from Salvia miltiorrhiza registered under CAS 90106-50-6 which is commercially available from many sources.

“Salvia miltiorrhiza” is a material used in traditional Chinese medicine. It is a complex extract comprising many compounds, including Salvianolic acid B, protocatechuic aldehyde, caffeic acid, rosmarinic acid, tanshinone, tanshinone IIA, tanshinone IIB, cryptotanshinone, tanshinone I, hydroxytanshinone, 3-hydroxytanshinone, methyl tanshinonate, tanshinaldehyde, dihydrotanshinone I, isotanshione IIA, isotanshione IIB, tanshinol A, tanshinol B, ferruginol, sibiriquinone A, sibiriquinone B, etc.

In a preferred embodiment the amount of Salvia miltiorrhiza extract in the composition of the invention is 19 ppm or more.

An embodiment of the invention is wherein the composition comprises an extract from Macrocystis pyrifera. Preferably the present invention relates to the use of a botanical extract from Macrocystis pyrifera registered under CAS 92128-82-0 which is commercially available from many sources.

Macrocystis pyrifera occurs in the eastern Pacific and the Southern Hemisphere. The extract is commonly used for flavoring purposes.

In a preferred embodiment the amount of Macrocystis pyrifera extract in the composition of the invention is 12 ppm or more.

### Method of Identifying an Extract for Use in the Treatment or Prevention of Viral Infection

A further aspect of the invention provides a method of identifying an extract for use in the treatment or prevention of viral infection comprising assaying whether an extract reduces viral entry into a cell using a cell-based pseudovirus entry assay.

Provided herein are methods by which the reduction of viral infection into a host cell can be measured. The use of such methods is known in the field of virology and can be readily adopted by the skilled person to determine if an extract reduces viral infection and hence is an extract which can be used in the composition of the invention.

In an embodiment of the invention, the method determines the IC₅₀ value of test extract and where that value is 7 ppm or less then the extract is considered to be an extract having use in the treatment or prevention viral infection.

The calculation of IC₅₀ measurements for botanical extracts can be readily performed by the skilled person using the methods disclosed herein and known in the art.

A further embodiment of the invention is wherein the method further comprises an ELISA assay or SPR assay of the extract.

Provided herein are methods by which the reduction of viral infection into a host cell can be measured using ELISA assay or SPR assay. Such assays can be used as a “prescreen” to identify extracts having possible viral infection inhibition potential prior to the cell-based pseudovirus entry assay.

### Compositions of the Invention

In an embodiment of the invention the composition of the invention may further comprise one or more of the following ingredients: antiviral agents, antibacterial agents, minerals, mineral salts, and/or vitamins, pharmaceutically acceptable excipients.

The composition of the invention may also comprise one or more antiviral agents.

Antiviral agents are drugs that inhibit the spread of virus, for example by preventing replication of the genome, blocking entry to host cells, or inhibiting viral protein synthesis or viral assembly.

Examples of such agents which can be used in the composition of the invention include: adamantane antivirals, antiviral boosters, antiviral combinations, antiviral interferons, chemokine receptor antagonists, integrase strand transfer inhibitors, miscellaneous antivirals, neuraminidase inhibitors, NNRTIs, NS5A inhibitors, nucleoside reverse transcriptase inhibitors (NRTIs), protease inhibitors and purine nucleosides.

The composition of the invention may also comprise one or more antibacterial agents. Suitable antibacterial agents include ethanol, triclosan, cetylpyridinium chloride (CPC) chlorhexidine (CHX), isopropyl methylphenol (IPMP), isopropyl methylphenol (IPMP), zinc sulfate (ZnSO₄), zinc citrate, zinc chloride (ZnCl₂).

In some embodiments of the present invention, compositions may further contain a mineral, mineral salt, or combinations thereof. Such minerals are not limited, and can include selenium, sulfur, zinc, iron, chlorine, cobalt, copper, manganese, molybdenum, and iodine.

The amount of the mineral or mineral salts in the topical formulation is not limited, and includes any therapeutically effective amount. For example, the mineral or mineral salt may have a concentration of about 0.01 wt. % to about 5 wt %, relative to the total amount of the composition, about 0.1 wt % to about 1 wt %, relative to the total amount of the composition, or any range or individual concentration encompassed by these example ranges.

In some embodiments of the present invention, the compositions may further include a vitamin or a combination of vitamins. Vitamins are organic molecules that are essential nutrients that organisms need to sustain proper biological function and metabolism. The vitamins encompassed by the invention are not limited, and can be, for example, vitamin A, vitamin B1, vitamin B2, vitamin B3, vitamin B4, vitamin B5, vitamin B6, vitamin B7, vitamin B8, vitamin B9, vitamin B10, vitamin B11, vitamin B12, vitamin C, vitamin D, vitamin E, and vitamin K.

The amount of the vitamin in the topical formulation is not limited, and can be any therapeutically effective amount. For example, the vitamin may have a concentration of about 0.01 wt. % to about 5 wt %, relative to the total amount of the composition, about 0.1 wt. % to about 1 wt %, relative to the total amount of the composition, or any range or individual concentration encompassed by these example ranges.

The composition of the invention may also comprise one or more pharmaceutically acceptable excipients.

The pharmaceutically acceptable excipients may be in the form of a solid, semi-solid, gelatinous, liquid, or combinations thereof. In certain embodiments, the composition is in an oral dosage form, such as a solid, semi-sold, gelatinous, or liquid oral dosage form. However, it is to be appreciated that the composition may be in any other dosage form known in the art.

When is a liquid dosage form, the pharmaceutically acceptable carrier may include water, oil, alcohol, or combinations thereof. The oral dosage form may be present in an amount of less than 100 milliliters. It is to be appreciated that powder/friable forms of the compositions and components may, for example, be mixed with food derived beverages, such as fruit juices, for oral administration by drinking the mixture.

When in a solid dosage form, the pharmaceutically acceptable carrier, for example, may be a compressed dosage form, such as a compressed tablet or caplet, obtained by compressing a powder mixture. The powder mixture may contain one or more active botanical extracts and optionally one or more excipients, such as binders, disintegrants, lubricants, fillers and the like, as known in the art.

### Preparations and Formulations of the Composition of the Invention

The compositions of the invention may be formulated in therapeutic form as a liquid, cream, lotion, liniment, ointment, gel, paste, tonic, tincture, oral pill, tablet, capsule, lip balm, spray or inhalant or combinations thereof.

Such compositions may be packaged in and/or administered via an appropriate pharmaceutical delivery system for delivery to the upper respiratory tract of a subject, such as an inhaler, a nebulizer, an atomizer, an oral spray, a nasal spray, or a dropper.

When administered in these formulations, including through the use of such delivery systems, these compositions may be applied to epithelial cells, including for example, respiratory epithelial cells, adenoid epithelial cells or bronchial epithelial cells of the subject being treated. Alternatively, or additionally, these compositions may be applied to endothelial cells of the subject being treated. The composition may be administered from one to four times a day or more, if indicated or needed for prevention or treatment.

In one embodiment, one or more of the components of the combination are provided as oral dosage forms. Oral dosage forms include tablets (coated or uncoated), capsules (hard or soft shell), caplets, pills, lozenges, syrups, solutions, powders, granules, elixirs and suspensions, sublingual tablets, wafers, or patches such as buccal patches. Oral dosage forms may also include sachets or stick packs.

Preferably, the compositions of the invention are provided as tablets.

Therefore, in one embodiment of the invention, at least one of the components (preferably all of the components) is presented in a tablet. In one embodiment, all of the components are presented in tablets, and in particular all components of the combination are presented in the same tablet i.e. the combination is administered in a unitary or fixed-dose.

Typically, the tablet includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavoring agents, sweeteners, taste masking agents, stabilizers or any other excipients conventionally used in pharmaceutical or nutraceutical compositions.

Preferably, the compositions of the invention are formulated with a pharmaceutically or nutraceutically acceptable filler or bulking agent.

Creams refer to semi-solid emulsions of oil and water in approximately equal proportions. They are divided into two types: oil-in-water (O/W) creams, composed of small droplets of oil dispersed in a continuous phase; and water-in-oil (W/O) creams, composed of small droplets of water dispersed in a continuous oily phase. Creams can provide a barrier to protect the skin. This may be a physical barrier or a chemical barrier as with UV-absorbing compounds. To aid in the retention of moisture (especially water-in-oil creams), creams are usually used for a variety of purposes including cleansing, emollient effects, and as a vehicle for drug substances such as local anesthetics, anti-inflammatoires (NSAIDs or corticosteroids), hormones, antibiotics, antifungals or counter-irritants.

Liniments or balms are topical formulations that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

Ointments are compositions in which oil and water are provided in a ratio of from 7:1 to 2:1, from 5:1 to 3:1, or 4:1. Ointments are generally formulated using oils, waxes, water, alcohols, petroleum products, water, and other agents to prepare formulations with various viscosities and solvent properties. Commonly used formulations include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

Lotions are low-to medium-viscosity topical preparation. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the composition can be in the form of a tincture. Tinctures are herbal extracts that provide a method for oral administration of an herbal component or components to a subject in need of treatment. Tinctures are prepared by mixing an herb or herbs or components and combinations thereof with a suitable solvent wherein a component or components of an herb or herbs or combinations thereof are extracted into a solvent in which the component or components of the herb are reasonably soluble. Suitable tincture solvents in the present invention include pharmacologically acceptable solvents such as organic solvents, water-based solvents, alcohols, and other orally administrable solvents such as, but not limited to, water, purified water, preserved water, vegetable glycerin, propylene carbonate, 3-methoxy-3-methyl-1-butanol (MMB), polyethylene glycol, glycerol, rice bran oil, and combinations thereof.

In some embodiments, the composition can be in the form of a tonic. Tonics are extracts that provide a method for oral administration of an herbal component or components to a subject in need of treatment. Tonics are prepared by mixing an herb or herbs or components and combinations thereof with a suitable solvent wherein a component or components of an herb or herbs or combinations thereof are extracted into a solvent by aid of heating, often heat necessary such that the solvent reaches its boiling temperature, in which the component or components of the herb are reasonably soluble. Suitable tonic solvents in the present invention include pharmacologically acceptable solvents such as organic solvents, water-based solvents, alcohols, and other orally administrable solvents such as, but not limited to, water, purified water, preserved water, vegetable glycerin, propylene carbonate, 3-methoxy-3-methyl-1-butanol (MMB), polyethylene glycol, glycerol, rice bran oil, and combinations thereof.

In some embodiments, the composition can be in the form of a capsule. Capsules generally fall within the class of either hard-shelled capsules or soft-shelled capsules, but need not be restricted to either class. Hard shelled capsules generally, but need not necessarily, contain dry, powdered, or granular components while soft-shelled capsules primarily, but need not necessarily, contain oils or medicaments or combinations thereof.

In one embodiment, one or more of the components of the combination are provided as oral dosage forms. Oral dosage forms include tablets (coated or uncoated), capsules (hard or soft shell), caplets, pills, lozenges, syrups, solutions, powders, granules, elixirs and suspensions, sublingual tablets, wafers or patches such as buccal patches. Oral dosage forms may also include sachets or stick packs.

Preferably, the compositions of the invention are provided as tablets.

Therefore, in one embodiment of the invention, at least one of the components (preferably all of the components) is presented in a tablet. In one embodiment, all of the components are presented in tablets, and in particular all components of the combination are presented in the same tablet i.e. the combination is administered in a unitary or fixed-dose.

Typically, the tablet includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavoring agents, sweeteners, taste masking agents, stabilizers or any other excipients conventionally used in pharmaceutical or nutraceutical compositions.

Preferably, the compositions of the invention are formulated with a pharmaceutically or nutraceutically acceptable filler or bulking agent.

Examples of excipients include dibasic calcium phosphate anhydrous, magnesium stearate, silicon dioxide, carboxymethylcellulose, crospovidone, hydroxypropyl cellulose and maltodextrin.

Preferably, the compositions of the invention are provided in capsules.

Therefore, in one embodiment of the invention, at least one of the components (preferably all of the components) is presented in a capsule. In one embodiment, all of the components are presented in capsules, and in particular all components of the combination are presented in the same capsule i.e. the combination is administered in a unitary or fixed-dose.

Typically, the capsule includes one or more pharmaceutically or nutraceutically acceptable excipient. The pharmaceutically or nutraceutically acceptable excipient can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilizers or any other excipients conventionally used in pharmaceutical or nutraceutical compositions.

Examples of excipients include dibasic calcium phosphate anhydrous, magnesium stearate, silicon dioxide, maltodextrin, carboxymethylcellulose, crospovidone, and hydroxypropyl cellulose.

Preferably, the compositions of the invention are provided as granulates.

Therefore, in one embodiment of the invention, at least one of the components (preferably all of the components) is presented as a granulate. In one embodiment, all of the components are presented in a granulate, and in particular all components of the combination are presented in a single granulate i.e. the combination is administered in a unitary or fixed-dose. The granulate may be packaged into a sachet or a stick pack.

The granulate may be prepared by dry or wet granulation techniques that are known in the art.

The composition of the invention may also be prepared and presented as an oral care product.

An embodiment of the invention is wherein the oral care product is selected from the group comprising of toothpaste, tooth powder, tooth gel, tooth cleaning liquid, tooth cleaning foam, mouth wash, mouth rinse, mouth spray, dental floss, chewing gum, dental aligners and lozenges.

As used herein, the term “oral healthcare composition” refers to a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral healthcare composition may be in various forms including toothpaste, dentifrice, tooth gel, subgingival gel, mouth rinse, solutions, mousse, foam, denture care product, mouth spray, lozenge or chewable tablet. The oral healthcare composition may also be incorporated onto floss, strips or films for direct application or attachment to oral surfaces or integrated into a device or applicator such as a toothbrush, dental aligners or roll-ons. Such applicators may be for single or multiple use.

As used herein, the term “dentifrice” includes paste, gel, or liquid formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

As used herein, the term “dispenser” refers to any pump, tube, or container suitable for dispensing compositions such as dentifrices.

### Consumer Products

An aspect of the invention provides a consumer product comprising a composition according to any of the previous claims.

The consumer product may be an “over the counter” product as known from pharmaceutical retail shops. Included here is where the consumer product is a cold remedy, lip balm, spry, ointment, therapeutic tonic drink, and nutraceutical products as known in the art.

Examples of consumer products which incorporate the composition of the invention also include, edible gel mixes and compositions, dental compositions, foodstuffs beverages and beverage products.

A non-exhaustive list of suitable form of the consumer product may include fried, frozen, marinated, battered, chilled, dehydrated, powder blended, canned, reconstituted, retorted, baked, cooked, fermented, microfiltred, pasteurized, blended or preserved.

Therefore, a flavored consumer product according to the invention comprises the invention's composition, as well as optional benefit agents, corresponding to taste and flavor profile of the desired edible product, e.g. a cream dessert.

Such consumer products may include further ingredients to enhance the consumer experience.

The consumer products may further include a flavoring agent. Examples of suitable flavoring agents include, but are not limited to, anethole, anise oil, benzaldehyde, blackberry, blueberry, caraway, caraway oil, cardamom oil, cardamom seed, cherry juice, cherry syrup, cinnamon, cinnamon oil, an alcohol, cinnamon water, citric acid, citric acid syrup, clove oil, cocoa, coriander oil, dextrose, eriodictyon, ethyl acetate, ethyl vanillin, fennel oil, ginger, glucose, glycerin, glycyrrhiza, grape, honey, lavender oil, lemon oil, lime, mannitol, methyl salicylate, myristica oil, orange oil orange peel, orange syrup, peppermint, peppermint oil, peppermint water, phenylethyl alcohol, pineapple, raspberry juice, raspberry syrup, rosemary oil, rose oil, rose water, sarsaparilla syrup, sorbitol, spearmint, spearmint oil, strawberry, sucrose, fructose, fruit juice, thyme oil, tolu balsam, vanilla, vanillin, wild cherry syrup, and combinations thereof.

Another component which can be part of a consumer products is a cooling agent and are generally used in amounts of 0.001% to 0.8% of the composition. Examples of cooling agents which can be used in the composition include menthol, p-menthanecarboxamide compounds such as N-ethyl-p-menthan-3-carboxamide, N,2,3-trimethyl-2-isopropylbutanamide, alcohol derivatives such as 3-1-menthoxy-propane-1,2-diol, isopulegol, p-menthane-3,8-diol; menthone glycerol acetal; menthyl esters such as menthyl acetate, menthyl acetoacetate, menthyl lactate, and monomenthyl succinate.

The consumer product is selected from the group comprising fabric care product, body-care product, cosmetic preparation, skincare product, air care product or home care product, or wherein the perfumery consumer product is a fine perfume, a splash or eau de perfume, a cologne, a shave or an after-shave lotion, a liquid or solid detergent, a fabric softener, a fabric refresher, an ironing water, a shampoo, a hair shaping product, a dental care product, a disinfectant, an intimate care product, a hair spray, a vanishing cream, a deodorant or antiperspirant, hair remover, tanning or sun product, nail products, skin cleansing, a makeup, a perfumed soap, shower or bath mousse, oil or gel, or a foot/hand care products, a hygiene product, an air freshener, a “ready to use” powdered air freshener, a mold remover, furnisher care, wipe, a dish detergent or hard-surface detergent, a leather care product, a car care product, or a product applied on face masks or other personal protective equipment (essential oil diffuser clips for face masks air freshener, etc.).

The consumer product is also selected from the group comprising beverages including alcoholic drinks (beer, cider, flavored alcoholic beverage, spirit, wine, etc.), carbonated soft drinks, other soft drinks (bottled water, flavored water, energy drink, functional drink, sport drink, etc.), Ready To Drink (tea, coffee, etc.), hot drinks (coffee, tea, etc.), juices and nectars, Powder Soft Drinks (fruit drink, tea, etc.); and sweet goods including bakery (biscuits, snacks, etc.), cereals, confectioneries (candies, jellies, tablets, gums, chewing gums, chocolate, etc.), dairies (milk, yoghurt and fruit preparations, dairy drinks, etc.), desserts (ice cream, fruits and preserves, etc.), dietary and nutrition (meal replacements, protein bars, protein drinks, etc.); and oral care products including toothpastes and mouthwashes; and savory goods including alternative proteins (cheese analogs, vegan proteins products, etc.), prepared meals (processed chesses, ready meals, etc.), snacks, meat proteins (processed meat, sausages, etc.), noodles and soups.

The invention will now be described in further detail by way of the following examples which illustrate the benefits and advantages of the present invention.

### EXAMPLES

### Example 1: Cell-Based Pseudovirus Entry Assay to Identify Botanical Extracts Interfering with Virus Activity

In general, pseudovirus entry assay is performed by using SARS-COV-2 (2019-nCOV) Spike pseudovirus (PSV001, Sino Biological) to infect human cell 293T-ACE2 that is over-expressing hACE2 on the cell surface (OEC001, Sino Biological). The SARS-COV-2 Spike pseudovirus (10¹⁰ virus copies/mL) is developed based on HIV backbone and packaged by SARS-COV-2 Spike S1 subunit (860 ng/ml), and it contains luciferase gene in the pseudovirus. It can bind to human cell 293T-ACE2 via SARS-COV-2 Spike S1 and enter cell and get its luciferase gene expressed by 293T-ACE2, so the Relative Light Unit (RLU) of luciferase (to around 106) that representing viral entry can be measured. (This pseudovirus entry assay is typically used for testing the existence of neutralizing antibody of SARS-COV-2 in serum samples). All extracts are solved in DMSO as 2% before use.

### Preparation:

1. The botanical extract samples are diluted with DMEM culture medium as 50, 25, 12.5, 6.25, 3.13, 1.56, 0.78, and 0.39 ppm, respectively.
2. The positive control, SARS-COV-2 neutralizing antibody, is diluted with DMEM culture medium as 100, 20, 4, 0.8, 0.16, 0.032, 0.0064, and 0.00128 μg/mL, respectively.

### Assay Procedure:

1. 293T-ACE2 cells are loaded to 96-well plate at 3×10⁴ cell/well in 100 μL of DMEM culture media containing 10% of Fetal Bovine Serum (FBS) and incubated in Water-Jacketed CO₂ Incubator at 37° C. with 5% CO₂.
2. 50 μL of SARS-COV-2 neutralizing antibody dilution (100, 20, 4, 0.8, 0.16, 0.032, 0.0064, and 0.00128 μg/mL, respectively) or botanical extract sample dilution (50, 25, 12.5, 6.25, 3.13, 1.56, 0.78, and 0.39 ppm, respectively) or negative control DMEM culture medium is pre-mixed with 50 μL of pseudovirus solution (10¹⁰ virus copies/mL).
3. The mixture is then loaded to the plated that has been prepared with 293T-ACE2 cells and incubated in Water-Jacketed CO₂ Incubator at 37° C. with 5% CO₂ for 48 hours. (Thus, the working concentrations of botanical extract sample dilutions is 12.5, 6.25, 3.13, 1.56, 0.78, 0.39, 0.2, 0.1 ppm, respectively.)
4. The RLU of each well is detected with Centro LB 960 Microplate Luminometer (Berthold Technologies)
   a) RLU value of the well that is only added with 100 μL of 293T-ACE2 cells and 100 μL of DMEM culture medium is set as RLU negative control.
   b) RLU value of the well that is added with 100 μL 293T-ACE2 cells, 50 μL DMEM culture medium and 50 μL of SARS-COV-2 Spike pseudovirus is set as RLU positive control.
   c) RLU value of the well that is added with 100 μL 293T-ACE2 cells, 50 μL botanical extract dilution (50, 25, 12.5, 6.25, 3.13, 1.56, 0.78, and 0.39 ppm, respectively) and 50 μL of SARS-COV-2 Spike pseudovirus is set as RLU sample test.
   d) RLU value of the well that is added with 100 μL 293T-ACE2 cells, 50 μL of SARS-COV-2 neutralizing antibody dilution (100, 20, 4, 0.8, 0.16, 0.032, 0.0064, and 0.00128 μg/mL, respectively) and 50 μL of SARS-COV-2 Spike pseudovirus is set as RLU antibody.
5. Data processing of the assay is calculated as below equations:

a) Inhibition rate % of botanical extract sample dilution=[1−(RLU sample test-RLU negative control)/(RLU positive control-RLU negative control)]×100%

b) Inhibition rate % of neutralizing antibody dilution=[1−(RLU antibody-RLU negative control)/(RLU positive control-RLU negative control)]×100%

The IC₅₀ (ppm or μg/mL) is calculated by following Reed-Muench method. And an average IC₅₀ is calculated based on the IC₅₀ of different batches of botanical extract samples.

### Example of Pseudovirus Entry Assay Results

| Common name, species name | IC₅₀ (ppm) |
|---|---|
| Blackcurrant, Ribes nigrum | 3.71 |
| Eucalyptus species, Eucalyptus globulus | 5.14 |
| Pine species, Pinus sylvestris | 4.44 |
| Black pepper, Piper nigrum | 0.97 |
| Turmeric, Curcuma longa | 1.76 |
| Laminaria, Laminaria digitata; and | 1.17 |
| Macrocystis, Macrocystis pyrifera | |
| Tea, Camellia sinensis | 6.73 |
| Cistus species, Cistus ladanifer | 2.63 |
| Artemisia species, Artemisia capillaris | 0.82 |
| Geum aleppicum | 2.13 |
| Salvia species, Salvia miltiorrhiza | 1.90 |

### Example 2: ELISA Screening to Identify Botanical Extracts Interfering with Viral Spike Binding to Receptor

In general, ELISA screening of interfering botanical extracts is performed by using SARS-CoV-2 (2019-nCOV) Inhibitor Screening ELISA Kit (KIT001, Sino Biological). All botanical extracts are solved in DMSO as 2% before use.

### Reagent Preparation: All the Solutions are Balanced at Room Temperature Before Use

1. 20 mL of 20×Wash Buffer Concentrate is added to distilled water to get 400 mL of 1×Wash Buffer.
2. 5 mL of 20×Dilution Buffer Concentrate is added to distilled water to prepare 100 mL of Dilution Buffer.
3. SARS-COV-2 Inhibitor is diluted with Dilution Buffer to 12, 4, 1.33, 0.44, 0.15, 0.05, 0.0165, 0.0055, 0.0018, and 0.0006 μg/mL.
4. Botanical extract samples are diluted with Dilution Buffer to 100, 50, 25, 12.5, and 6.25 ppm.
5. hACE2 (His Tag) recombinant protein is prepared by adding 1 mL of Dilution Buffer to a stock (2 mg/mL), then 1:40 diluted with Dilution Buffer before use.
6. Anti-His Tag Ab (HRP) is 1:3000 diluted with Dilution Buffer before use.
7. Substrate Solution is prepared by mixing Color Reagents A and B as 1:1 within 10 minutes of use, protected from light. 200 μL of the resultant mixture is required per well.

### Assay Procedure:

1. 300 μL of Wash Buffer is used to wash the wells of the microplate (pre-coated by SARS-COV-2 Spike RBD at 0.5 μg/mL) and completely removed by decanting and blotting the plate against clean paper towels, repeated by 3 times.
2. 100 μL of SARS-COV-2 Inhibitor dilution (12, 4, 1.33, 0.44, 0.15, 0.05, 0.0165, 0.0055, 0.0018, and 0.0006 μg/mL, respectively) or botanical extract sample dilution (100, 50, 25, 12.5, and 6.25 ppm, respectively) is added per well, then 100 μL of hACE2 (50 ng/ml, His Tag) is added to each well. The microplate is sealed and incubated for 1 hour at room temperature. (Since the incubation is 1:1 mixture of extract sample dilution and hACE2, the working concentrations of botanical extract sample dilutions in the data processing step is set 50, 25, 12.5, 6.25, and 3.13 ppm, respectively.)
3. 300 μL of Wash Buffer is used to wash the wells of the incubated microplate and completely removed by decanting and blotting the plate against clean paper towels, repeated by 3 times.
4. 100 μL of Anti-His Tag Antibody (HRP) in working concentration is added to each well. The microplate is sealed and incubated for 1 hour at room temperature.
5. 300 μL of Wash Buffer is used to wash the wells of the incubated microplate and completely removed by decanting and blotting the plate against clean paper towels, repeated by 3 times.
6. 200 μL of Substrate Solution is added to each well. The microplate is incubated for 15 min at room temperature, protected from light.
7. 50 μL of Stop Solution is added to each well and mixed well.
8. The optical density of each well is detected within 15 minutes using a microplate reader (Multiskan MK3, Thermo) set to 450 nm.
   a) The optical density of the well that is sequentially incubated with hACE2 (His Tag) and Anti-His Tag Antibody (HRP) is set as background OD450.
   b) The optical density of the well that is sequentially incubated with botanical extract sample dilution (100, 50, 25, 12.5, and 6.25 ppm, respectively) and hACE2 (His Tag) and Anti-His Tag Antibody (HRP) is set as sample test OD450.
   c) The optical density of the well that is sequentially incubated with SARS-COV-2 Inhibitor dilution (12, 4, 1.33, 0.44, 0.15, 0.05, 0.0165, 0.0055, 0.0018, and 0.0006 μg/mL, respectively) and hACE2 (His Tag) and Anti-His Tag Antibody (HRP) is set as positive control OD450.
9. Data processing of the assay is calculated as below equations:

a) Binding rate of botanical extract sample dilution=sample test OD450/background OD450×100%

b) Binding rate of SARS-COV-2 Inhibitor dilution=positive control OD450/background OD450×100%
The IC₅₀ (ppm) of each of botanical extract sample is thus calculated with a dose (botanical extract sample dilutions)—response (binding rates) curve.

### Example of ELISA Screening Results

| Common name, species name | IC₅₀ (ppm) |
|---|---|
| Blackcurrant, Ribes nigrum | 78.25 |
| Eucalyptus species, Eucalyptus globulus | 62.34 |
| Pine species, Pinus sylvestris | Not significant |
| Black pepper, Piper nigrum | 53.38 |
| Turmeric, Curcuma longa | 10.46 |
| Laminaria species, Laminaria digitata; and | 7.52 |
| Macrocystis, Macrocystis pyrifera | |
| Tea, Camellia sinensis | 101.03 |
| Cistus species, Cistus ladanifer | 135.32 |
| Artemisia species, Artemisia capillaris | 5.03 |
| Geum aleppicum | 6.80 |
| Salvia species, Salvia miltiorrhiza | 8.58 |

### Example 3: SPR Assay of Botanical Extract from Pine (Pinus sylvestris)

SPR assay of botanical extracts is performed by using Biacore T200 (GE Healthcare). In general, SARS-COV-2 Spike RBD (1.78 mg/mL, Sino Biological) is fixed on GE Healthcare Biacore™ Sensor Chip CM5. hACE2 protein (0.26 mg/mL, Sino Biological) is used as analyte.

### Assay Procedure:

1. EDC/NHS is used to balance Sensor Chip CM5 for 5 minutes.
2. SARS-COV-2 RBD is diluted to 5 μg/mL by using 10 mM sodium acetate (pH4.5) and a total amount of 1200 RU of SARS-COV-2 Spike RBD is fixed on Sensor Chip CM5.
3. 10 mM HEPES (pH8.0,150 mM NaCl, 0.05% P20) is as running buffer. Baseline signal is record by Biacore T200.
4. When analyzing, analyte hACE2 is diluted in the running buffer with concentrations 0, 1.625, 3.25, 6.5, 13, 26 μg/mL (corresponding to 0, 19.1, 38.2, 76.4, 153, 306 nM). Analyte in different concentrations is pumped in by 30 μL/min, last for 3 minutes, so the hACE2 is bind to SARS-COV-2 Spike RBD on CM5. Then running buffer is pumped in for 7 minutes to dissociate the binding between hACE2 and SARS-COV-2 Spike RBD. The signal curve is recorded by Biacore T200, which is used to calculate k on (1/Ms), k off (1/s), and KD (M) as control data.
5. In the screening, botanical extract is pumped with analyte to measure the change on k on (1/Ms), k off (1/s), and KD (M).

### Example of SPR Assay Results

| | | | Botanical | | | |
|---|---|---|---|---|---|---|
| Test name | Fixed phase | Analyte | extract | k on (1/Ms) | k off (1/s) | KD (M) |
| Pine | SARS-CoV-2 | hACE2 | Pine species, | 191000 | 0.0247 | 1.3E⁻⁰⁷ |
| | Spike RBD | | (Pinus sylvestris) | | | |
| Control | SARS-CoV-2 | hACE2 | | 50800 | 0.00311 | 6.1E⁻⁰⁸ |
| | Spike RBD | | | | | |

The KD_{Pine} is 2 folds of KD_{control}, indicating that Pinus sylvestris extract interferes the binding between hACE2 and SARS-CoV-2 RBD.

### Example 4: Zeta Potential of Botanical Extracts

In general, zeta potential of the extract is examined by using a Zetasizer Nano ZS (Malvern). All botanical extracts are solved in DMSO as 2% before use. The extracts are diluted into water phase (deionized water) with pH at 7.0±0.2. Then the dilution was put into a cell for measurement under constant temperature (25° C.).

### Example of Zeta Potential Measurements

| Common name, species name | Zeta potential (mV) |
|---|---|
| Blackcurrant, Ribes nigrum | −18.4 ± 1.04 |
| Eucalyptus species, Eucalyptus globulus | −11 ± 0.493 |
| Pine species, Pinus sylvestris | −17.5 ± 0.473 |
| Black pepper, Piper nigrum | −23.8 ± 0.208 |
| Turmeric, Curcuma longa | −17.1 ± 2.35 |
| Laminaria species, Laminaria digitata; and | 2.13 ± 0.688 |
| Macrocystis, Macrocystis pyrifera | |
| Tea, Camellia sinensis | −6.22 ± 1.19 |
| Cistus species, Cistus ladanifer | −11.5 ± 2.1 |
| Artemisia species, Artemisia capillaris | −4.96 ± 0.272 |
| Geum aleppicum | −3.71 ± 0.447 |
| Salvia species, Salvia miltiorrhiza | −0.186 ± 0.207 |

### Example 5: Cell-Based Pseudovirus Entry Assay of Flavors Containing Botanical Extracts

A combined extract from Laminaria and Macrocystis species, in addition Camellia sinensis extract, are considered as suitable components in tea formulation and thus formulated in a simple tea flavor for further testing the interfering effect. The combined extract from Laminaria and Macrocystis species is used at 1.2 ppm and the Camellia sinensis extract is used at 18 ppm.

Pseudovirus entry assay is performed as described in Example 1. The working concentration of Laminaria and Macrocystis species is 0.3 ppm in the assay, whereas the working concentration of Camellia sinensis in the assay is 4.5 ppm. Data processing of the assay is calculated as below equations:

$$Inhibition rate \% of flavor = [ 1 - ( RLU flavor - RLU negative control ) / ( RLU positive control - RLU negative control ) ] \times 100 \%$$

The rest results are in below table. Flavor ID 1 inhibited 80% of viral entry.

| Flavor ID | Laminaria species | | Camellia sinensis | | Inhibition rate |
|---|---|---|---|---|---|
| 1 | 0.3 | ppm | 4.5 | ppm | 80.10% |
| 2 | 0 | ppm | 0 | ppm | −58.13% |

### Example 6: Cell-Based Pseudovirus Entry Assay of Botanical Extracts Against Spike of SARS-COV-2 Delta Variant B.1.617.2

In general, pseudovirus entry assay is performed by using SARS-COV-2 Spike pseudovirus of delta variant B.1.617.2 (PSV011, Sino Biological) to infect human cell 293T-ACE2 that is over-expressing hACE2 on the cell surface (OEC001, Sino Biological). The SARS-COV-2 Spike pseudovirus of delta variant B.1.617.2 (10¹⁰ virus copies/mL) is developed based on Spike of delta variant B.1.617.2 and it contains luciferase gene in the pseudovirus. It can bind to human cell 293T-ACE2 via Spike S1 of delta variant B.1.617.2, and enter cell and get its luciferase gene expressed by 293T-ACE2, so the Relative Light Unit (RLU) of luciferase (to around 106) that representing cell entry can be measured. All botanical extracts are solved in DMSO as 2% before use.

### Preparation:

1. The botanical extract samples are diluted with DMEM culture medium as 50, 25, 12.5, 6.25, 3.13, 1.56, 0.78, and 0.39 ppm, respectively.
2. The positive control, SARS-COV-2 neutralizing antibody, is diluted with DMEM culture medium as 100, 20, 4, 0.8, 0.16, 0.032, 0.0064, and 0.00128 μg/mL, respectively.

### Assay Procedure:

1. 293T-ACE2 cells are loaded to 96-well plate at 3×10⁴ cell/well in 100 μL of DMEM culture media containing 10% of FBS and incubated in Water-Jacketed CO₂ Incubator at 37° C. with 5% CO₂.
2. 50 μL of SARS-COV-2 neutralizing antibody dilution (100, 20, 4, 0.8, 0.16, 0.032, 0.0064, and 0.00128 μg/mL, respectively) or botanical extract sample dilution (50, 25, 12.5, 6.25, 3.13, 1.56, 0.78, and 0.39 ppm, respectively) or negative control DMEM culture medium is pre-mixed with 50 μL of pseudovirus solution (10¹⁰ virus copies/mL).
3. The mixture is then loaded to the plated that has been prepared with 293T-ACE2 cells and incubated in Water-Jacketed CO₂ Incubator at 37° C. with 5% CO₂ for 48 hours. (Thus, the working concentrations of extract sample dilutions is 12.5, 6.25, 3.13, 1.56, 0.78, 0.39, 0.2, 0.1 ppm, respectively.)
4. The RLU of each well is detected with Centro LB 960 Microplate Luminometer (Berthold Technologies)
   a) RLU value of the well that is only added with 100 μL of 293T-ACE2 cells and 100 μL of DMEM culture medium is set as RLU negative control.
   b) RLU value of the well that is added with 100 μL 293T-ACE2 cells, 50 μL DMEM culture medium and 50 μL of SARS-COV-2 spike pseudovirus of delta variant B.1.617.2 is set as RLU positive control.
   c) RLU value of the well that is added with 100 μL 293T-ACE2 cells, 50 μL botanical extract dilution (50, 25, 12.5, 6.25, 3.13, 1.56, 0.78, and 0.39 ppm, respectively) and 50 μL of SARS-COV-2 spike pseudovirus of delta variant B.1.617.2 is set as RLU sample test.
   d) RLU value of the well that is added with 100 μL 293T-ACE2 cells, 50 μL of SARS-COV-2 neutralizing antibody dilution (100, 20, 4, 0.8, 0.16, 0.032, 0.0064, and 0.00128 μg/mL, respectively) and 50 μL of SARS-COV-2 spike pseudovirus of delta variant B.1.617.2 is set as RLU antibody.
5. Data processing of the assay is calculated as below equations:

a) Inhibition rate % of botanical extracts sample dilution=[1−(RLU sample test-RLU negative control)/(RLU positive control-RLU negative control)]×100%

b) Inhibition rate % of neutralizing antibody dilution=[1−(RLU antibody-RLU negative control)/(RLU positive control-RLU negative control)]×100%

The IC₅₀ (ppm or μg/mL) is calculated by following Reed-Muench method.

### Example of Pseudovirus Entry Assay Results

| Common name, species name | IC₅₀ (ppm) |
|---|---|
| Blackcurrant, Ribes nigrum | 6.38 |
| Eucalyptus species, Eucalyptus globulus | 4.63 |
| Pine species, Pinus sylvestris | 0.41 |
| Black pepper, Piper nigrum | 11.77 |
| Laminaria species, Laminaria digitata; and Macrocystis, | 5.53 |
| Macrocystis pyrifera | |
| Cistus species, Cistus ladanifer | 10.15 |

The invention claimed is:

1. A composition for use in the prevention or treatment of a viral infection, wherein the composition comprises an extract, wherein said extract:
(i) is a botanical extract;
(ii) reduces viral infection into a host cell, with an IC₅₀ of 10 ppm or less; and,
(iii) has a zeta potential of 10 mV or less.

2. The composition of claim 1, wherein the composition comprises an extract selected from the group consisting of Pine species, Pinus sylvestris, Pinus virginiana, Pinus thunbergia, Eucalyptus species, Eucalyptus globulus, Eucalyptus maidenii, Eucalyptus smithii, Ribes nigrum, Cistus species, Cistus ladanifer, Cistus ladanifer subsp. Mauritianus, Cistus ladanifer subsp. sulcatus, Cistus palhinhae, Cistus criticus, Cistus laurifolius, Piper nigrum, Laminaria species, Laminaria digitata, Laminaria japonica, Laminaria ephemera, Curcuma longa, Camellia sinensis, Camellia sinensis var. assamica, Artemisia species, Artemisia capillaris, Artemisia scoparia, Artemisia dracunculus, Artemisia nakaii, Artemisia pubescens, Geum aleppicum, Salvia species, Salvia miltiorrhiza, and combinations thereof.

3. The composition of claim 1, wherein the composition comprises an extract having a zeta potential of −10 mV or less.

4. The composition of claim 3, wherein the composition comprises an extract selected from the group consisting of Pine species, Pinus sylvestris, Pinus virginiana, Pinus thunbergia, Eucalyptus species, Eucalyptus globulus, Eucalyptus maidenii, Eucalyptus smithii, Ribes nigrum, Cistus species, Cistus ladanifer, Cistus ladanifer subsp. Mauritianus, Cistus ladanifer subsp. sulcatus, Cistus palhinhae, Cistus criticus, Cistus laurifolius, Piper nigrum, Curcuma longa.

5. The composition of claim 1, wherein the virus infection is caused by a coronavirus.

6. The composition of claim 4, wherein the coronavirus is SARS-CoV-2.

7. The composition of claim 1, wherein the extract is at a concentration of 10 ppm or more.

8. The composition of claim 1, wherein said composition further comprises one or more of the following ingredients: antiviral agents, antibacterial agents, minerals, mineral salts, vitamins, and pharmaceutically acceptable excipients.

9. The composition of claim 1, wherein said composition is prepared in a therapeutic form as a liquid, cream, lotion, liniment, ointment, gel, paste, tonic, tincture, oral pill, tablet, capsule, lip balm, oral spray, nasal spray or inhalant, or a combination thereof.

10. A consumer product comprising a composition according to claim 1.

11. The consumer product of claim 10, wherein the consumer product is selected from the group consisting of fabric care product, body-care product, cosmetic preparation, skincare product, air care product and home care product, or wherein the consumer product is a perfumery consumer product selected from the group consisting of fine perfume, a splash or eau de perfume, a cologne, a shave or an after-shave lotion, a liquid or solid detergent, a fabric softener, a fabric refresher, an ironing water, a shampoo, a hair shaping product, a dental care product, a disinfectant, an intimate care product, a hair spray, a vanishing cream, a deodorant or antiperspirant, hair remover, tanning or sun product, nail products, skin cleansing, a makeup, a perfumed soap, shower or bath mousse, oil or gel, foot/hand care products, a hygiene product, an air freshener, a “ready to use” powdered air freshener, a mold remover, furnisher care, wipe, a dish detergent or hard-surface detergent, a leather care product, a car care product, a product applied on face masks or other personal protective equipment, and a product applied on essential oil diffuser clips for face masks or air freshener.

12. The consumer product of claim 10, wherein the consumer product is selected from the group consisting of beverages, alcoholic drinks, beer, cider, flavored alcoholic beverage, spirit, wine, carbonated soft drinks, non-carbonated soft drinks, bottled water, flavored water, energy drink, functional drink, sport drink, Ready To Drink drinks, tea, coffee, hot drinks, juices, nectars, Powder Soft Drinks, fruit drink, sweet goods, bakery goods, biscuits, snacks, cereals, confectioneries, candies, jellies, tablets, gums, chewing gums, chocolate, dairies, milk, yoghurt and fruit preparations, dairy drinks, desserts, ice cream, fruits and preserves, dietary and nutrition, meal replacements, protein bars, protein drinks, and oral care products, toothpastes and mouthwashes, savory goods, alternative proteins, cheese analogs, vegan proteins products, prepared meals, processed chesses, ready meals, snacks, meat proteins, processed meat, sausages, noodles, and soups.

13. A method of treatment or prevention of viral infection, the method comprising applying to the oral and/or nasal cavity of a person in need thereof a composition according to claim 1.

14. A method of identifying an extract for use in the treatment or prevention viral infection, the method comprising assaying whether an extract reduces viral entry in a cell using a cell-based pseudovirus entry assay.

15. The method of claim 14, wherein the method further comprises an ELISA or a SPR assay of the extract.
